(19) United States
(12) Patent Application Publication
(10) Pub. No.: US 2006/0260606 A1
(43) Pub. Date: Nov. 23, 2006

(54) Inhalable formulations of amphotericin B and methods and devices for delivery thereof

(76) Inventor: Robert E. Coifman, Millville, NJ (US)

Correspondence Address: Licata & Tyrrell P.C., 66 E. Main Street, Marlton, NJ, 08053, US

(21) Appl. No.: 11/131,964

(22) Filed: May 18, 2005

(51) Int. Cl. A61M 11/00

(52) U.S. Cl. 128/200.14

(57) ABSTRACT

An inhalable formulation of amphotericin B and a hydrofluoroalkane propellant in for pressurized metered dose aerosol canisters is provided. Also provided are methods for administering amphotericin B to the upper or lower respiratory tract of a patient with this inhalable formulation and a pulsatile nasal administration device for delivery of medications packaged in a metered dose aerosol canister to the paranasal sinuses.

### FIELD OF THE INVENTION

The present invention relates to formulations of amphotericin B as either a solution or a suspension in a hydrofluoroalkane propellant for administration to the upper and/or lower respiratory tract of a patient via a pressurized metered dose inhaler. The present invention also relates to methods for administering these amphotericin B formulations to the upper and/or lower respiratory tract for treatment of various conditions including but not limited to chronic sinusitis and asthma. The present invention also relates to a pulsatile nasal administration device for paranasal delivery of this amphotericin B formulation and other drugs.

### BACKGROUND OF THE INVENTION

Amphotericin B is an antifungal polyene antibiotic obtained from a strain of Streptomyces nodosis. Amphotericin B is a highly lipophilic molecule, insoluble in water. Three suspension dosage forms of amphotericin are manufactured for intravenous infusion, each either complexed with various phospholipids or incorporated into the walls of small liposomes. All are designed for slow intravenous administration following dilution with either water or 5% dextrose, and cannot be mixed with saline as the suspended drug comes out of suspension in the presence of salt. These products were formulated for intravenous administration to treat systemic fungal infections in patients in which the potential benefit and lack of less toxic alternatives justify a significant risk of adverse drug effects.

Attempts have also been made to treat diseases caused or believed to be caused by susceptible fungi in accessible portions of the respiratory tract by topical application of available or compounded amphotericin B formulations in an effort to alleviate adverse side effects associated with systemic administration of amphotericin B. For example, amphotericin B has been administered by nebulizer to treat bronchopulmonary fungal infections (Marra et al. Annals of pharmacotherapy 2002 36(1):46-51; Lambros et al. J. Pharm. Sci. 1997 86(9):1066-9; and Diot et al. European Respiratory Journal 1995 8(8):1263-8). An aqueous amphotericin B suspension has also been administered nasally as a stream of drug directed toward the maxillary sinus based upon the hypothesis that chronic rhinosinusitis may be caused in part by an abnormal hypersensitivity reaction to fungi identified in the allergic mucin from chronic sinusitis patients (Ponikau J U et al. Mayo Clinic Proceedings 1999; 74:877-884) (Ponikau et al. Journal of Allergy & Clinical Immunology 2002;110:862-866; Ponikau et al. Journal of Allergy & Clinical Immunology 2005; 115:125-131). Similar studies performed in an open label trial demonstrated amphotericin B to bind to membrane lipids on cells involved in nasal polyposis (an extreme variant of hyperplastic rhinosinusitis) in the same way in which it binds to the cell membranes of fungi (Ricchetti et al. The Journal of Laryngology and Otology 2002:116:261-263). Further, Ricchetti et al. suggest that the therapeutic effect of amphotericin B in chronic rhinosinusitis may result from its direct effect on nasal tissues rather than from activity against fungi (The Journal of Laryngology and Otology 2002:116:261-263).

To achieve effective delivery of an inhaled aerosolized drug to the distal parts of the lung, the drug must be inhaled according to a procedure that maintains linear airflow, as turbulent flow results in drug loss by impaction on the surfaces of the proximal airway. In 1968, Kauf reported the opposite side of the same phenomenon, namely adding additional turbulence to nasal airflow by means of pressure fluctuations and vibrations increased lateral impaction of inhaled aerosols sufficiently to cause penetration of and deposition within the paranasal sinuses (Kauf H. Eindringvermögen von Aerosolen in Nebenraume 1968;190(1):95-108). WO 2004/20029 discloses a nebulizer device exploiting this phenomenon for delivery to the paranasal sinuses of any of a broad range of drugs including amphotericin B, if formulated for aerosolization from jet nebulizers (i.e., in few milliliter volumes of solution or suspension in pharmaceutically acceptable aqueous vehicles). This PCT application discloses a traditional jet nebulizer modified by either connection to or incorporation of a piston-driven membrane or other pressure-generating device to superimpose pressure fluctuations in the frequency range of 10 to 100 Hz on continuous airflow into the nebulizer that aerosolizes the medication and propels it through a snugly fitting nose-piece into the user's nose. However, it is disclosed that with the above system, in which pressure fluctuations are superimposed on the continuous flow of air into the nebulizer, it is necessary to create a back-pressure by requiring the patient to block airflow from the back of the nose to the throat by voluntary elevation of the soft palate, and further by partially obstructing outward airflow through the opposite nostril. Such requirements limit use of this technique to patient populations able to perform this maneuver.

Diseases which are potential targets for topical application of amphotericin B in the respiratory system include allergic and non-allergic rhinitis and rhinosinusitis, chronic hyperplastic rhinosinusitis with or without nasal polyposis, topically accessible infections with susceptible fungi in the nasal cavity and paranasal sinuses, asthma and related inflammatory conditions of the lower respiratory tract which are pathophysiologically similar to allergic and non-allergic rhinosinusitis, and topically accessible infections with susceptible fungi in the lower respiratory tract. Presently available lipid complex and liposomal suspension formulations of amphotericin B have distinct pharmaceutical liabilities for topical delivery to these sites whether by instillation, spraying or nebulization. This is because these lipid suspensions are stable in either water or sugar solutions but the lipid particles agglomerate and come out of suspension in solutions containing salt. Each of the conditions these formulations are intended to treat is aggravated by edema at the site of drug action. Edema of airway target tissues increases the obstruction and congestion that contribute to morbidity and resistance to treatment of each of the above described diseases. Stable aqueous suspensions of amphotericin B in water are hypo-osmotic. When applied topically, their water content is rapidly absorbed by iso-osmotic tissues on contact, thus increasing edema of the very tissues in which edema must be controlled if treatment is to be successful. Sugars as osmotic agents have the liabilities of supporting proliferation of airborne microbial organisms in multi-dose containers even if handled in ways to minimize gross contamination, and if present in formulations applied to the respiratory tract they serve as nutrients to promote microbial growth where they land. Salt is the most physiologic and least toxic osmotic agent for topical application to the respiratory tract but destabilizes lipid suspensions as has been noted.

Pressurized metered dose aerosol canisters have been proven to be a convenient, effective and user-friendly means to package a variety of pharmaceutical products for delivery to the respiratory tract, by spraying the aerosol directly onto target tissues in the nose, by spraying aerosol into the open mouth for inhalation, or by discharging aerosol into a “spacer” or “holding chamber” (exemplified by the Aerochamber, from Trudell Medical International, London, Ontario, Canada) from which the aerosol is then inhaled. Drugs delivered to the respiratory tract from metered dose aerosol canisters have included adrenergic bronchodilators, topically acting corticosteroids, anticholinergics and such non-steroid inhibitors of allergic mediator release as cromolyn sodium and nedocromil. Since the global cessation of manufacture of chlorofluorocarbon propellants because of their destructive effect on the ozone layer, hydrofluoroalkanes have become the propellants of choice for metered dose aerosol canisters for human respiratory drug delivery.

### SUMMARY OF THE INVENTION

An object of the present invention is to provide an inhalable formulation of amphotericin B comprising a solution or suspension of amphotericin B and a hydrofluoroalkane propellant in a pressurized metered dose aerosol canister.

Another object of the present invention is to provide a method for administering amphotericin B to the upper or lower respiratory tract of a patient in need thereof which comprises administering to the patient an inhalable formulation of amphotericin B comprising a solution or suspension of amphotericin B and a hydrofluoroalkane propellant via a pressurized metered dose aerosol canister.

Another object of the present invention is to provide a pressurized metered dose aerosol canister containing a solution or suspension of amphotericin B and a hydrofluoroalkane propellant.

Another object of the present invention is to provide a holding chamber for aerosolized drug discharged from a pressurized metered dose aerosol canister, said holding chamber equipped with a pump to deliver pulses of air mixed with aerosolized drug to the nose with sufficient turbulence to achieve paranasal sinus delivery.

### BRIEF DESCRIPTION OF THE FIGURE

FIG. 1 provides a diagram of representative components and geometries of a device for pulsatile nasal administration of amphotericin B or any other drug appropriately packaged in a pressurized metered dose inhaler to the paranasal sinuses without need for placement of a catheter, by using pulsed airflow to generate turbulence.

### DETAILED DESCRIPTION OF THE INVENTION

The present invention relates to an inhalable formulation of amphotericin B, packaged in and administered from a pressurized metered dose aerosol canister.

In simplest form, the inhalable formulation comprises a solution or suspension of amphotericin B. Amphotericin B is designated chemically as [1R(1R*,3S*,5R*,6R*,9R*,11R*,15R*, 16R*,17R*,18S*,19E,21E,23E,25E,27E,29E,31E,33R*,35S*,36R*,37 S*)]-33-[(3-amino-3,6-dideoxy-β-(D-mannopyranosyl)oxy)-14,39-dioxabicyclo[33,3,1]nonatriaconta-19,21,23,25,27, 29,31-heptaene-36-carboxylic acid. Compositions of amphotericin B for reliable and reproducible optimal metered dose inhaler deliver preferably comprise drug particles in the range from approximately 1 to 70 microns mass median aerosol diameter and contain necessary quantities of pharmaceutically acceptable co-solvents, surface active agents, dispersing agents, preservatives, lubricants and other additives.

The inhalable formulation further comprises a liquefied or compressed gas which acts as the propellant. Preferred are hydrofluoroalkane propellants. Various hydrofluoroalkanes, also referred to as hydrofluorocarbons, have been developed for use as substitutes to the ozone damaging chlorofluorocarbon propellants. Different hydrofluoroalkanes or mixtures of hydrofluoroalkanes can be used as propellants in the formulations of the present invention. Examples of hydrofluoroalkane propellants useful in the present invention include, but are in no way limited to, 1,1,1,2-tetrafluoroethane (HFA 134a) and 1,1,1,2,3,3,3-heptafluoropropane (HFA227) and mixtures thereof. See, for example, U.S. Pat. No. 6,713,047, teachings of which are herein incorporated by reference in their entirety. Choice of propellant mixture among the various hydrofluoroalkanes, may also effect whether the drug is in suspension or a solution.

The resulting mixture of amphotericin B suspension or solution and hydrofluoroalkane propellant of the present invention can be administered by a hydrofluoroalkane-powered metered dose inhaler without the counterproductive osmotic effect of distilled water vehicle or other hypo-osmotic aqueous vehicle, or the infection-promoting effect of a dextrose-containing vehicle.

Various metered dose inhalers for use in the present invention are known. Such metered dose inhalers conventionally consist of a pressurized container having a metering valve of fixed volume which will measure individual doses of the amphotericin B suspension or solution from the container. For accuracy in dosing any suspended amphotericin B must be homogeneously and consistently dispersed in the container. Further, the valve performance must be reproducible and effective throughout the time during which the inhaler is in use. On depressing the valve stem of the metering valve, the propellant fraction of the metered dose is rapidly vaporized so as to aerosolize the amphotericin B, which is then either inhaled by the user or expelled into a holding chamber from which it is pumped in the recipient's nostril in small pulses to create the turbulence needed for sinus penetration and deposition. An exemplary metered dose inhaler useful in the present invention is described in U.S. Pat. No. 6,737,044, teachings of which are incorporated herein by reference in their entirety. However, as will be understood by those of skill in the art upon reading this disclosure, other conventional metered dose inhalers can be used.

With variation in propellant composition and valve chamber design, for example as taught in U.S. Pat. No. 6,737,044, it is possible to vary particle size from a range appropriate for direct delivery to the nose or to the maxillary or other large sinuses via semi-rigid catheters positioned and left in place for intervals of treatment, to a smaller particle size range suitable for inhalation into the distal portions of the lung or for efficient retention in a holding chamber for delivery to the paranasal sinuses by turbulent nasal airflow. Particle sizes appropriate for direct delivery to the nose or to the maxillary or other large sinuses via semi-rigid catheters positioned and left in place for intervals of treatment range from approximately 7 to 70 microns mass median aerosol diameter. Smaller particles suitable for inhalation into the distal portions of the lung or for efficient retention in a holding chamber for delivery to the paranasal sinuses by turbulent nasal airflow range in size from approximately 1 to 5 microns mass median aerosol diameter. Formulations of the present invention can be used in treatment of chronic sinusitis without the liabilities of aqueous formulations that are either hypo-osmotic or osmotically stabilized with dextrose or other sugars. Formulations of the present invention may be inhaled into the lungs for treatment of bronchopulmonary infections with susceptible fungi. Furthermore, if the apparent beneficial effect of topically applied amphotericin B in chronic rhinosinusitis reflects metabolic and reactive responses common to the physiologically similar mucosal tissues of the upper and lower airway, it is expected that formulations of the present invention with particle sizes appropriate for lower airway delivery will also be beneficial in asthma.

Administration of amphotericin B twice daily via inhalation dosing for periods of months to years is expected to be useful in treating chronic hyperplastic rhinitis, rhinosinusitis, and asthma. The duration of treatment for specific fungal infections is dependent on the nature of the infections and on the presence of factors increasing that patient's susceptibility to that infection. Dosages in the range of from about 0.5 mg per nostril to about 5 mg per nostril are expected to be effective and safe for treatment of nasal and sinus diseases. Effective doses for the lower respiratory tract are dependent on target tissue delivery efficiency, which can be optimized with programmed inhalation and breath-holding. See e.g., Clark et al, J. Aerosol Med. 2003; 16(2):188.

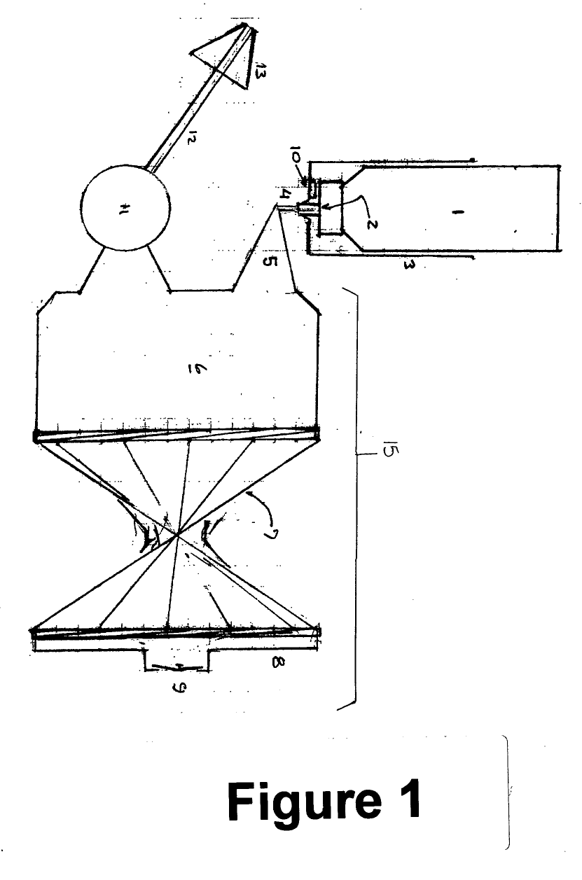

Also provided in the present invention is a holding chamber for administration of a drug from a pressurized metered dose aerosol canister, that incorporates a pump to deliver pulses of drug-containing aerosol to the nose and by means of this pulsed airflow generate the turbulence needed to achieve paranasal sinus deposition of the amphotericin B formulation of the present invention as well as other drugs. An exemplary embodiment of this pulsatile nasal administration device is depicted in FIG. 1. The intranasal turbulence generated by the pulsed delivery of drug-containing aerosol from the holding chamber results in paranasal sinus drug deposition without need for placement of a catheter.

As shown in FIG. 1, the device for pulsatile nasal administration of a drug aerosol is sized to fit a drug canister 1 with a tip 2, which when depressed after the drug canister 1 is gently shaken and held in a vertical position actuates an internal valve 10 of the holding chamber. An exemplary embodiment of an internal valve 10 useful in the present invention is taught in U.S. Pat. No. 6,863,195 teachings of which are herein incorporated by reference in their entirety. When the drug canister 1 is gently shaken and placed in the actuator sleeve 3, downward pressure forces the tip 2 against the actuator nozzle 4 releasing a mist of drug through the outflow cone 5 into the holding chamber 15. In one embodiment, as depicted in FIG. 1, the holding chamber 15 comprises both a fixed portion 6 and an expandable portion 7 which expands conically in the illustrated embodiment of FIG. 1, to accommodate the bolus of drug aerosol released from the canister 1.

By “expands, conically” it is meant a portion of the holding chamber 15 that is essentially cylindrical when expanded, and which twists and folds as it collapses. As it twists, a point that is, for example, at the top of the distal portion of the chamber when fully expanded may rotate approximately 120° downward as the cylinder collapses. The portions of the surface of the holding chamber that remain straight are the geometrical equivalent of rays or ribs, so that as the cylinder collapses the ray that extends, for example, across the top of the holding chamber, when expanded, extends from the top to a position 120° below the top when collapsed, thereby narrowing the middle of the cylinder into an hourglass shape with conical walls, in which the ends also move closer together as the sides collapse inward. See FIG. 1. Alternate embodiments may have chambers that are completely expandable (except for their valves and fittings), or, alternatively, completely fixed.

In the embodiment of FIG. 1, the far wall 8 of the holding chamber 15 forms the rear wall of the expandable portion 7 and has an air inlet with a one way valve 9 positioned to allow low resistance airflow into the holding chamber 15 even when the expandable portion 7 is in its collapsed position. In some embodiments, it is desirable to provide a small resistance to airflow into the holding chamber through the air inlet with a one way valve 9. The purpose of this resistance is to ensure that the expandable portion of the holding chamber collapses to its minimum washout volume as air is pumped out of it and into the nose. In simplest form, this is accomplished by providing a narrow orifice through which air must flow before it can enter the holding chamber through the air inlet with one way valve 9. A pump 11 such as a small battery-operated electromagnetic pump delivers pulses of aerosol from the holding chamber 15 into an outflow channel 12 ending with a nosepiece 13 wedged snugly into the user's nostril. The pump 11 may be turned on by downward pressure of the canister 1 against a switch 14 on the internal valve 10 as it is pressed downward to activate and release drug. Alternatively, the pump may be turned on by a sensor located in the pressurized metered dose inhaler along the path from the actuator nozzle 4 through the outflow cone 5 into the holding chamber 15 that signals when a dose of drug is released into the holding chamber 15.

The device of the present invention can be used to deliver amphotericin formulations as well as other inhalable drug formulations.

In one embodiment, the device can be used to deliver a fine particle aerosol (1 to 2.5 micron mass median aerosol diameter). Delivery of a fine particle aerosol has advantages in comparison to aerosols with larger particles because fine particles remain airborne for a longer time, with less loss of drug by settling out, in the holding chamber.

For both user convenience and to minimize drug loss by settling out in the holding chamber, it is desirable that dosing time be less than or equal to approximately 15 seconds if each nostril must be treated separately, or about 30 seconds if sufficient turbulence can be maintained through both sides of the nasal airway to treat both sides of the nasal cavity while instilling drug into only one. For an exemplary device for pulsatile nasal administration of a drug aerosol of the present invention with a fixed portion of the holding chamber having a volume of 50 ml and an expandable portion having a volume varying from 10 to 100 ml, if one wants to achieve washout of the original expanded volume of the chamber (150 ml) plus two fills of the residual 60 ml collapsed volume in 15 seconds, the necessary rate of airflow is 270 ml per 15 seconds or 1.08 liters per minute.

Both static pressure and the vibratory pulse pressure that generates the turbulence that drives drug into the paranasal sinuses decrease along the pressure gradient from entrance to exit of the nasal cavity. The entrance is where the holding chamber output enters the nostril through a nosepiece 13, and the exit is either the nasopharynx (if one nostril is being treated and the patient is allowing air from the back of the nostril to exit into his or her throat) or the other nostril (if the patient has closed that exit by voluntary elevation of the soft palate so that the vibrating air column enters through one nostril and exits through the other). Depending on the interaction between the various design parameters of the system and the patient's nasal airway resistance, it may be necessary to add back-pressure by adding resistance to airflow out of the opposite nostril (opposite to the one receiving aerosol) to maintain pressure and turbulence within the nasal airway. This can be accomplished most directly by placing a high resistance stopper in the opposite nostril.

The optimal pulse frequency for the device of the present invention ranges from 5 to 100 Hz, more preferably 10 to 55 Hz.

Totally pulsed airflow into the nose using this dosing device provides a more efficient generator of turbulence than steady inflow modulated by a superimposed pressure oscillation such as described in WO 2004/20029. Further, a higher rate of pulsed nasal airflow more effectively maintains pressure as the aerosol traverses the nasal airway. It is believed that the device of the present invention will provide a sufficiently high rate of pulsed nasal airflow to achieve and maintain sufficient unilateral nasal turbulence thereby providing good drug deposition into the paranasal sinuses without need for voluntary closure of the posterior nasal airway by elevation of the soft palate. Thus, the pulsatile nasal administration device of the present invention is expected to be particularly useful in successful sinus drug delivery in young children and others who cannot perform the maneuver of closing the nasopharynx.

What is claimed is:

1. An inhalable formulation of amphotericin B comprising a solution or suspension of amphotericin B and a hydrofluoroalkane propellant in a pressurized metered dose aerosol canister.

2. A method for administering amphotericin B to the upper or lower respiratory tract of a patient in need thereof comprising administering to the patient the inhalable formulation of amphotericin B of claim 1.

3. The method of claim 2 wherein the patient is suffering from chronic sinusitis or asthma.

4. A device for pulsatile nasal administration of a drug aerosol which creates turbulence needed for effective penetration of the drug into the paranasal sinuses comprising a holding chamber for administration of a drug from a pressurized metered dose aerosol canister with a pump that delivers pulses of drug-containing aerosol to a user's nose to generate turbulence needed to achieve paranasal sinus deposition of the drug.
